(19) United States
(12) Patent Application Publication
(10) Pub. No.: US 2009/0198217 A1
(43) Pub. Date: Aug. 6, 2009

(54) Convenience IV kits and methods of use

(75) Inventors: Gale H. Thorne, JR., Bountiful, UT (US); Gale H. Thorne, Bountiful, UT (US)

Correspondence Address: Gale H. Thorne, 1056 Millcrest Circle, Bountiful, UT, 84010, US

(73) Assignee: Gale H. Thome, JR., Bountiful, UT (US)

(21) Appl. No.: 12/319,326

(22) Filed: Jan. 6, 2009

Related U.S. Application Data
(63) Continuation-in-part of application No. 12/080,185, filed on Apr. 1, 2008.
Application No. 12/080,185 is a continuation-in-part of application No. 12/012,837, filed on Feb. 6, 2008.

(51) Int. Cl. A61M 5/31

(52) U.S. Cl. 604/518; 604/83

(57) ABSTRACT

Two convenience kits are disclosed. A first convenience kit provides a basic configuration for use in measuring, filling and dispensing medication and flush solutions to IV sets and patient catheters through needleless connectors while improving safety and efficacy by requiring fewer post-sterilization makes and breaks compared to conventional filling and dispensing methods. A second convenience kit which, being a companion to the first convenience kit, provides safety in access to a vial. Methods of use of both kits improve flush compliance by facilitating dispensing of flush solutions and decreases likelihood of infections by providing for flushing of connecting sites while reducing numbers of breaks required for associated medical procedures.

### CONTINUATION-IN-PART

This Application for Patent is a Continuation-in-Part of U.S. patent application Ser. No. 12/080,185, filed, Apr. 1, 2008, which is a Continuation-in-Part of U.S. patent application Ser. No. 12/012,837 filed Feb. 6, 2008, all three of which are made part of this Application by reference.

### FIELD OF INVENTION

This invention relates to medical intravenous administration of fluids, specifically for medical applications including push or bolus and drip (from a hanging container) dispensing. It is also particularly related to kits and to methods which employ preassembled parts which are substantially fabricated for the purpose of achieving a significant decrease in need for making and breaking line connections and other product manipulations and for reducing dangerous conditions related to administering hazardous drugs.

### BACKGROUND AND DESCRIPTION OF RELATED ART

Recently, increased use of needleless IV connectors has resulted in a generation of problems and procedures related to IV safety. Recognition of some IV system use problems has resulted in the following principles, considerations and guidelines:

A basic principle taught in IV therapy is that every IV delivered medication should be flushed. Flushing of an IV administration port, such as a Y-injection site on an IV set, associated with a catheter helps prevent incompatible drug mixing and assure delivery of a timely, complete dose and decreases likelihood of drug contamination by residual drops or wetted surfaces on the outside of the port. Recognition of a need to clear a “Y-site” following injection of a dose through a port has led to a widespread practice of drawing in flush from an available saline source (such as a hanging bag which communicates with the receiving catheter); however, this practice was shown to be questionable as disclosed in U.S. patent application Ser. No. 12/313013 filed Nov. 14, 2008, from which this patent application continues-in-part.

Unfortunately, many nurses forget to flush or assume that a running IV line will flush a Y-injection site thereby leaving small amounts of medication in the Y-site where incompatible drug mixing may occur. Also, an undesirably high catheter replacement rate in central lines may be a direct consequence of a failure to consistently flush lines.

A Jul. 5, 2005, PHC4 Research Brief entitled “Hospital-acquired Infections in Pennsylvania” reported that clinician-caused (nosocomial) bloods infection rates in Pennsylvania may be as high as 21,458 per year at a treatment cost of $861 million and mortality rate of 25.6% in 2004 alone. Such treatment costs in hospitals extrapolate to a $20.3 billion cost and over 80,000 deaths per year in the United States. Additional studies that cite similar increases in infection rates led to the “100,000 lives Campaign” instigated by the Institute for Healthcare Improvements, Cambridge, Mass. Clinicians who work in IV therapy are well schooled in knowing that “the more line breaks (disconnections), makes (connections) and line manipulations, the greater the chance for contamination.” Reducing line breaks, makes and line manipulations, in principle, will reduce line contaminations and patient infections. Please note that a simple connection of a flush syringe after disconnection of a dose administration syringe adds an additional make and two additional breaks (clinician must remove a cap on the flush syringe) to perform a procedure.

A chronic nursing shortage, projected to persist beyond 2012, places nursing time at a premium. Short-staffed healthcare facilities result in busier nurses who may be more prone to medical errors, some of which result in serious consequences for patients. A product which would save nursing time by reducing nursing steps would simplify care-giver procedures and by reducing nursing steps may also reduce clinician errors and overall healthcare costs.

A 2004 NIOSH (National Institute of Occupational Safety and Health) Safety Alert: Preventing Occupational Exposure to Antineoplastics and Other Hazardous Drugs in Healthcare Settings warns healthcare institutions about the need to provide products and procedures to protect clinicians from hazardous drug exposure. Attempts to reduce such drug exposure has resulted in use of expensive protective port attachment devices.

Thus, there exists a severe contemporary need for devices, not currently available commercially, which reduce injection site makes and breaks, reduce nurse time, facilitate ease of flushing and provide a greater degree of safety related to line contamination and subsequent patient infection and care-giver risk to hazardous drug exposure.

### Terms and Definitions

In the following table 1 is a list of terms and associated definitions provided to improve clarity and understanding of precepts of the instant invention:

TABLE 1
bolus, adj: a type of medication delivery to a patient, i.e. through a syringe
break, n: a disconnection of a pair of medical connectors, as part of a medical procedure.
clip, n: a holder for a pair of syringes for stabilizing the syringes.
dead space, n: a volume of inaccessible fluid, retained within a device after a procedure.
extension set, n: any tubing and associated connecting parts which provide ports used for
fluid medication delivery through a catheter.
dose syringe, n: an initially empty syringe which is filled with a prescribed dose of
medication.
flush syringe, n: a syringe, pre-filled with a predetermined volume of flush solution.
half-life, n: a period of time during which drug activity or usefulness declines by half
fitting, n: a medical connector for fluids.
IV set, n: intravenous drug delivery tubing specifically dedicated for use with an
associated IV catheter and IV container.
IV container, n: a container, made of glass or plastic in the form of a bottle or IV bag used
to hold and deliver IV fluids containing a saline solution and/or other medications for
delivery through an IV set to a patient.
kit, n: a group of parts, provided within a single package for a designated medical use
luer fitting, n: a medical connector which is in common use in medical practice.
luer lock fitting, n: a luer fitting having a locking mechanism whereby a male and female
connector are securely, but releasibly affixed one to the other.
make, n: a connection or re-connection of a pair of medical connectors.
multi-dosing, v: action of drawing more than one dose of medication from a single vial
needleless connector, n: a fitting which permits needle free fluid access to an IV set or
through a vial adapter and which has interface geometry similar to a conventional syringe
port, n: a site for a medical connector, where through fluid is communicated
pouch, n: a bag or tray.
short extension set, n: tubing and associated connecting parts used for connecting a fluid
valve to a pre-filled syringe.
subkit, n: a group of parts provided as a unit within a kit (used alone, a subkit is a kit).
TPA, n: one of a set of drugs used for clearing blood-clot occluding catheters.
unitized, adj: a plurality of separate parts permanently joined to be used as a single unit.
wrap, n: a flexible container which may be a bag or folded shield which is sealed to
provide a cover in which enclosed parts are sterilized and protected until opened for use.

### BRIEF SUMMARY AND OBJECTS OF THE INVENTION

In brief summary, use of this novel invention generally decreases known problems related to makes and breaks and resultant IV line contaminations, enumerated supra, while increasing patient safety when dealing with catheter related injection ports and associated devices. The inventive concept involves providing kits which are used with other components or parts, generally available at an assembly site, to construct a medical assembly which can be used to substantially reduce inadvertent risk of contamination of hazardous drugs and of infection due to makes and breaks during drug administration. Each kit comprises a fluid switching component As disclosed in earlier patent applications from which this patent application continues, one kit also includes a short extension set as the basis for a two syringe (i.e. a dose syringe and a flush syringe) assembly, and other basic parts of the assembly disclosed in detail hereafter, which are used to improve safety and efficacy of drug administration.

Use of these kits resolves a number of issues related to conserving nursing and pharmacist time. Each invention is a dedicated convenience kit comprising a pouch or wrap containing parts, which are sterilized therein, with some parts preferably unitized, and which are assembled with other readily available parts, at an assembly site, preparatory to performing a medical procedure involving delivery of medication through an IV set. Each of these convenience kits may be used with other convenience kits assembled with additional parts for specific medical procedures. Generally, kits may be used in two stages, (1) preparation (usually in pharmacy) and (2) delivery (at site of use).

### Kit 1

In a first kit, components comprise a fluid switching component, a short extension set and other items specifically made available for kit use and not readily available at a preparation site. The fluid switching component has attachment sites for access to at least two syringes which become part of the assembled kit apparatus when affixed thereto. The fluid switching component has another attachment site through which filling and dispensing fluids from and to, respectively, an external source is performed. It should be noted that a simple luer attachment, as specified for male luers in general, may not be sufficient at this site because, though all male luer attachments provide connecting geometry which is necessary for connecting to luer ports, not all male luer attachments provide the necessary geometry required to reliably connect to a needleless connector.

As retaining purity of contents of both dose and flush syringes is critical in most applications involving Kit 1, the fluid switching component inherently keeps contents of the dose and flush syringes disparate until fluid from each syringe is dispensed into a receiving connector through the other attachment site. Further, it is important that consideration be given to deterring any reflux flow into a flush syringe of such a kit assembly.

As stated supra, it is important that the other attachment site interfaces reliably with all IV set and injection port configurations. It is particularly important that a reliable connection be made with needleless fittings, in general.

As two syringes (dose and flush) are used in tandem during a sequence of drug and flush dispensing, preferably with the use of but a single hand, it is important that both syringes be presented to a user in a manner which is conducive to single-hand operation. For this purpose, the short extension set is provided to permit orienting the flush syringe parallel relative to the dose syringe. Further, a clip provides opportunity to stabilize the syringes for such use.

At a station where Kit 1 components and other items are assembled for use (for example, in pharmacy where a syringe is filled with a prescribed drug.) a dose syringe and a flush syringe are affixed to the kit components. Preferably the station is in a controlled environment (such as in a sterile area and/or under a laminar flow hood) so that kit components may be accessed yet remain contamination free. It is preferred that kit components be provided to a preparer in a “ready to use” format which will not inadvertently come apart.

One of the compelling purposes of convenience kits resulting from this invention is providing an inherently associated flush syringe. As contents of a flush syringe should, in most cases, be kept disparate from a prescribed drug prior to drug delivery, it is important that a secure fluid switching component be used to controllably regulate filling and delivery pathways. For this purpose, it is presently preferred to use a stopcock. Even so, other modes of fluid regulation may be used within the scope of the invention. As an example, “Y” sites with clamps on extensions of tubing therefrom may be used. Also, other switching components, as disclosed herein, may be used so long as disparate and fitting compatibility criteria are met.

Stopcocks are commonly used in medical practice; however, a stopcock configuration for at least one convenience kit application (for hazardous drugs such as those used in oncology) is not generally available commercially. Disclosure of such a stopcock is provided in detail hereafter. Once a dose syringe is filled in the pharmacy using the present invention, preferably using a negative pressure technique, a pharmacist may switch the stopcock to enable flushing by the present invention of the disconnecting parts prior to disconnection. Thus, is enabled a safety break at a flushed site in the system when disconnecting from a vial adaptor.

Once preparation in pharmacy is complete (e.g. the dose syringe prescription is attached and filled and stopcock/vial adapter flushed) and a flush syringe is affixed to the kit components, with exit pathways capped and protected, the assembled kit components should be labeled and packaged for transport to the site of use following institutional protocol. At a patient delivery site of use, for example, contents of the package are removed and after removing the cap, with but a single make, connected to an IV set dispensing port whereat, using the stopcock as the switching mechanism, the dose syringe is emptied as prescribed, followed by flush delivery of a remaining flush to assure compliance with guidelines for flushing.

Handling two syringes affixed to a stopcock may require a fixture to stabilize one of the syringes while using the other. For such purposes, a dual syringe clip is an element of the instant invention provided to facilitate syringe handling.

One example of a Kit 1 assembly, based upon the present invention, is a hazardous drug kit assembly. While nearly all drugs may be considered to be somewhat hazardous, such drugs as anti-neoplastic drugs used in oncology are particularly dangerous. For example, some anti-neoplastic drugs are considered extremely dangerous, even if contact is made simply upon skin as a liquid or inhaled as a vapor.

To alleviate the likelihood of exposing a hazardous drug to an environment outside a drug filled syringe, the presence of a pre-filled flush syringe as part of a kit assembly provides a unique opportunity for safety. In this case, the pre-filled syringe and drug dispensing syringe are connected to a common dispensing pathway through a fluid switching component (e.g. a stopcock, where the stopcock is used as the fluid switching component). The stopcock, or any other switching component used according to this invention, should be designed and constructed to permit only one communicating pathway from one of the syringes at a time. Thus, after the syringe is filled with drug in stage 1 (state one of the switching component), the pathway from the dose syringe to the dispensing pathway is closed to the dose syringe and afterward opened to the flush syringe (state two of the switching component). Then, a predetermined amount of flush liquid is dispensed through the dispensing pathway to flush drug from the dispensing pathway and leave flush liquid at the attachment site, as disclosed supra Similarly in stage 2, after a desired drug volume has been dispensed from the dose syringe (in state one of the switching component), a desired amount of flush liquid is dispensed through the dispensing pathway and through an attached catheter to displace potentially harmful reagents from both the associated catheter and connection port (in state two of the switching component) prior to disconnecting kit parts from the catheter injection port or an associated IV set.

Another advantage of a kit 1 assembly made according to the instant invention is found when administering a short half-life drug (e.g. adenosine). Short half-life drugs, administered through a catheter, must be delivered to their target organ in as short a time as possible. In such cases, it is common practice to connect two syringes to two different “Y” injection sites on an IV set connected to a patient catheter to permit delivery of the short half-life drug from one syringe handled by a first care-giver, followed by delivery of flush from a second syringe by a second care-giver. Having both the dose syringe and flush syringe available to a single dispensing pathway, through a stopcock or other switching device, provides opportunity for a single care-giver to dispense the short half-life drug, switch the dispensing pathway and immediately dispense the flush syringe. Using the syringe stabilizing clip permits simple motion of a thumb from one syringe plunger stem to the other, while switching the stopcock, to change syringe dispensing modes.

### Kit 2

A second kit (Kit 2) is a companion to Kit 1 and provides for safety and more efficiency in preparation of IV containers in Pharmacy and in handling multi-dosing. Kit 2 simply comprises a path selection device such as a special stopcock (which is different from the stopcock disclosed for Kit 1). Such a stopcock may be a two-way stopcock, having pathway switching control whereby only pathways between two female connectors and between a dedicated dose syringe pathway and a male luer fitting (the two female connectors and male luer fitting being commonly part of medical stopcocks, in general). The stopcock is unitized to a vial adapter and may be further affixed to a needleless connector at one of the stopcock female connectors.

Kit 2 is used in a plurality of ways to acquire medication from a medical drug vial and delivery of that drug to a site of use or provide a pathway to flush unwanted hazardous drug into a safety waste receptacle. Of significant importance is using Kit 2 to deliver hazardous drug contents from a medical vial to an IV container through associated length of catheter connecting tubing. While other parts may be interfaced to accomplish a method of filling, using Kit 1 is particularly efficacious for transferring medical fluids from a vial to a service point. Service points include filling components of Kit 1 for bolus delivery and filling an IV container for transfer fluid within a substantially closed system. Thus, there are two general applications or methods of use for Kit 2 parts.

A first application involves providing a closed environment for transferring contents of a vial to an IV control flow container (e.g. a drip delivery container). Contents of Kit 2 are affixed to a needleless connector of the associated stopcock and further connected to an IV port where through access is provided to the target IV control flow container. In this case, using parts of Kit 1 and preferably negative pressure procedures, a predetermined volume of medicant is transferred from the vial to a dose syringe (of Kit 1) with the pathway switching device (of Kit 2) set in a first state. Then the pathway switching device of Kit 2 is switched to a second state and the medicant is delivered through the IV port toward the IV container. The pathway switching device of Kit 1 is the switch to provide a pathway for a flush solution to be delivered to the IV port. The port and container access is then flushed with a predetermined volume of liquid. Thus, a predetermined volume of liquid is transferred through a closed system to the IV container. Safety breaking connection to the IV port is achieved after the flushing step.

Of course, consideration should be given to reconstituting lyophilized contents of a vial prior to transfer. In such a case, liquid must be injected into such a vial (it is highly preferred to make such liquid transfers under negative pressure) for reconstitution. Such may be achieved using negative pressure transfer techniques and supplying reconstitution liquid from the flush syringe.

Once a medicant is in a liquid state, medicant may be drawn and measured into a dose syringe (e.g. a dose syringe affixed to Kit 1 parts) as disclosed supra and delivered to an IV port and associated IV container in a first application or retained in the dose syringe for ultimate bolus delivery from the Kit 1 assembly in a second application. In either case, each disconnection site is flushed to provide a safety breaking point.

For the second application, the needleless connector is displaced to the other female connector of the Kit 2 pathway selection device to provide a disconnection point between a fluid delivery system provided by parts of Kit 2 and Kit 1. A refuse container (e.g. a syringe or a bag with a luer fitting) is affixed to the female connector of the pathway switching device of Kit 2, previously affixed to the needleless connector. This refuse container remains affixed to the second kit parts and disposal is made of the entire Kit 2 assembly and refuse container as a single unit as specified by institutional protocol.

Preparation for bolus delivery using an assembled and filled Kit 1 assembly is performed by affixing a Kit 1 assembly to a Kit 2 assembly via the needleless connector affixed to a female fitting of the pathway switching device of Kit 2. Medicant is drawn into the dose syringe from an attached source vial. The connecting point associated with the needleless connector is flushed, using contents of the flush syringe, with delivery of excess flushed liquids to the refuse container affixed to the other female fitting of the Kit 2 switching device. Thus, the needleless connector attachment point is flushed and cleared so separation may be made thereat and Kit 1 assembly detached from the Kit 2 assembly for delivery of the Kit 1 assembly to a site of use.

Accordingly, it is a primary object to provide methods and apparatus for preparing and using convenience kits for intravenous medical applications.

It is an object to provide methods and apparatus for preparing and using convenience kits for intravenous delivery of hazardous drugs.

It is an object to provide methods and apparatus for preparing and using convenience kits for intravenous delivery of short half-life drugs.

It is a very important object to provide a first kit which provides access for two syringes.

It is also a very important object of a first kit to provide an attachment site from a fluid switching component for a dose filling and dispensing syringe as one of the two syringes.

It is yet another very important object of the first kit to provide an attachment site from a fluid switching component for a pre-filled flush or pre-fillable flush syringe as one of the two syringes.

It is a compelling object of the first kit to provide, for selectively controlling the pathways, a switching component, affixed to each syringe, which provides a single pathway therefrom.

It is a more compelling object of the first kit to provide a switching component which assures that fluid within each syringe is kept disparate from fluid within the other syringe.

It is a still more compelling object to provide a fluid switching component having a single input/output pathway, for fluids dispensed from either a dose syringe or a flush syringe, which is geometrically and functionally compatible with general requirements for a needleless connecting port on an IV set or vial access device.

It is an important object of the first kit to provide a fluid-switching component which obstructs reflux flow through the fluid-switching component into the flush syringe.

It is a meaningful object of the first kit to provide a clip for stabilizing the two syringes for single handed operation of the apparatus.

It is another meaningful object of the first kit to provide a clip which may be used with syringes of various syringe barrel diameters.

It is a critical object to provide kits for constructing assemblies which significantly reduce makes and breaks required for a predetermined procedure to lessen likelihood of contamination associated with such makes and breaks in a conventionally performed procedure.

It is another critical object of kits according to inventions of this application that such adjoined parts be unreleasibly affixed (unitized) to preclude separation in transport and storage.

It is a further important object to provide a second kit and associated method for acquiring fluid from a vial and dispensing it directly to an IV container without an associated make or break during such transfer.

It is an object to provide a second kit and method for acquiring and delivering multiple doses from a vial to individual IV containers with safety.

It is a still further object to provide a second kit and associated method for delivering multiple doses from a vial to dose syringes affixed to separate first kit assemblies with safety.

These and other objects and features of the present invention will be apparent from the detailed description taken with reference to accompanying drawings.

### BRIEF DESCRIPTION OF THE DRAWINGS

FIG. 1 is a perspective of a dual syringe assembly, comprising two syringes, a stopcock and a syringe clip, configured according to a first kit of the instant invention.

FIG. 2A is a schematic drawing of a prior art three-way stopcock having three connecting ports and a rotatable core having a handle disposed to show a closed port thereat

FIG. 2B is a schematic drawing of the three-way stopcock seen in FIG. 2A with the core and handle rotated to close a second port.

FIG. 2C is a schematic drawing of the three-way stopcock seen in FIGS. 2A and 2B with the core and handle rotated to close a third port.

FIG. 3A is a schematic drawing of a two-way stopcock found commonly in commerce and which is similar in structure and position to the three-way stopcock seen in FIG. 2A, but having stops which restrict core rotation (and port closures) to two positions, the first port closure position being seen in FIG. 3A.

FIG. 3B is a schematic drawing of the two-way stopcock seen in FIG. 3A with the core and handle rotated to close a second port.

FIG. 4A is a schematic drawing of a two-way stopcock having a core and handle and associated stops configured according to a first kit of the present invention and closing the port indicated on the handle.

FIG. 4B is a schematic drawing of the two-way stopcock seen in FIG. 4A, but with the core and handle rotated to close another port.

FIG. 4C is a perspective of a stopcock and an associated short extension set portion of the assembly seen in FIG. 1.

FIG. 4D is a magnified perspective of the stopcock and a portion of the associated extension set seen in FIG. 4C.

FIG. 4E is a front elevation of a PRIOR ART stopcock.

FIG. 4F is a front elevation of a stopcock made in accordance with the first kit of the present invention.

FIG. 5A is a schematic drawing of another two-way stopcock having a core and handle and associated stops configured according to the first kit of the present invention and closing the port indicated on the handle.

FIG. 5B is a schematic drawing of the two-way stopcock seen in FIG. 5A, but with the core and handle rotated to close a different port.

FIG. 6A is a schematic drawing of a dual syringe and stopcock assembly with the stopcock core rotated to permit dispensing from a first syringe.

FIG. 6B is a schematic drawing of the dual syringe and stopcock assembly seen in FIG. 6A with the stopcock core rotated to permit dispensing for a second syringe.

FIG. 7A is a front elevation of a dual syringe holder or clip associated with the first kit, as disclosed supra.

FIG. 7B is a front elevation of the dual syringe holder or clip seen in FIG. 7A with a cross section of syringe barrels inserted into the clip.

FIG. 8 is a front elevation of another syringe holder or clip having a pair of syringe holding cavities, each cavity having a pattern which could hold one of three different barrels of three different syringe sizes.

FIG. 9 is a perspective of a presently preferred dual syringe holder or clip.

FIG. 10 is a perspective of a dual syringe assembly of the first kit, comprising two syringes, a stopcock and the syringe clip seen in FIG. 9, the assembly being configured according to the instant invention, with an oversized syringe barrel disposed in one side of the clip.

FIG. 11 is a perspective of a group of parts assembled for use in a first kit according to the present invention.

FIG. 12 is a perspective of the parts, seen in FIG. 11, packaged for sterilization and shipment.

FIG. 13 is a perspective of a dual syringe/stopcock assembly configured according to the first kit of the present invention with the stopcock disposed for filling a preselected syringe.

FIG. 14 is a perspective of the dual syringe/stopcock assembly seen in FIG. 13, but wherein the predetermined syringe has been filled and stopcock reoriented to permit dispensing of fluid from the other syringe.

FIG. 15 is a perspective of the dual syringe/stopcock assembly seen in FIGS. 13 and 14, but with the stopcock disposed for dispensing fluid from the predetermined syringe.

FIG. 16 is a perspective of the dual syringe/stopcock assembly seen in FIGS. 13-15, but with the stopcock oriented for dispensing fluid from the other syringe.

FIG. 17 is a perspective of an assembly according to the instant invention which incorporates a pressure operated fluid switch assembly made in accordance with the first kit of the present invention.

FIG. 18 is a perspective of the assembly seen in FIG. 17 with a tube clamp closing a tube pathway from the pressure operated fluid switch to an associated flush syringe.

FIG. 19 is a cross section of the pressure operated fluid switch assembly which may be used as seen in FIGS. 17 and 18, the switch assembly being disposed in a state providing a pathway from a dose syringe connection to a common output pathway.

FIG. 20 is a cross section of the pressure operated fluid switch assembly seen in FIG. 19, the switch assembly being disposed in a state providing a pathway from a flush syringe connection to the common output pathway.

FIG. 21 is a cross section of the pressure operated fluid switch assembly seen in FIG. 20, the switch assembly being disposed in a state wherein pathways from the flush syringe connection and dose syringe connection to the output pathway are blocked.

FIG. 22 is a cross section of another pressure operated fluid switch assembly which may be used as seen in FIGS. 17 and 18, this switch assembly being disposed in a state providing a pathway from a dose syringe connection to a common output pathway.

FIG. 23 is a cross section of the pressure operated fluid switch assembly seen in FIG. 22, the switch assembly being disposed in a state providing a pathway from a flush syringe connection to the common output pathway.

FIG. 24 is a cross section of the pressure operated fluid switch assembly seen in FIG. 22, the switch assembly being disposed in a state wherein pathways from the flush syringe connection and dose syringe connection to the output pathway are blocked.

FIG. 25 is a side elevation of a PRIOR ART IV set.

FIG. 26 is a side elevation of an IV set having an inverted Y-site port affixed and inferiorly disposed relative to a saline containing bag and associated spike of the IV set.

FIG. 27 is a graph of a concentration gradient associated with disposition of a medical syringe being used in a flush mode.

FIG. 28 is a graph of the concentration gradient, seen in FIG. 27, displaced about a connector and a Y-site.

FIG. 29 is a graph of a concentration of dispensed medicine about the connector and Y-site illustrated in FIG. 28.

FIG. 30 is a graph of a concentration gradient about the connector and Y-site seen in FIG. 29 following a saline flush from a pre-filled flush syringe associated with the assembly seen in FIG. 1.

FIG. 31 is an exploded view of parts associated with the second kit of the present invention.

FIG. 32 is a side elevation of a first assembled configuration of the second kit seen in FIG. 31.

FIG. 33 is a side elevation of a second assembled configuration of the second kit seen in FIG. 31.

FIG. 34 is a side elevation of the assembled configuration of the second kit seen in FIG. 33, with an associated stopcock core rotated.

FIG. 35 is a schematic of parts associated with the first and second kits assembled for transfer of fluid from an associated vial to a dose syringe affixed to the first kit

FIG. 36 is a schematic of parts associate with the first and second kits assembled for transfer of fluid from an associated vial to an IV container (e.g. bag) affixed through a needleless connector to the second kit.

### DETAILED DESCRIPTION OF THE ILLUSTRATED EMBODIMENTS

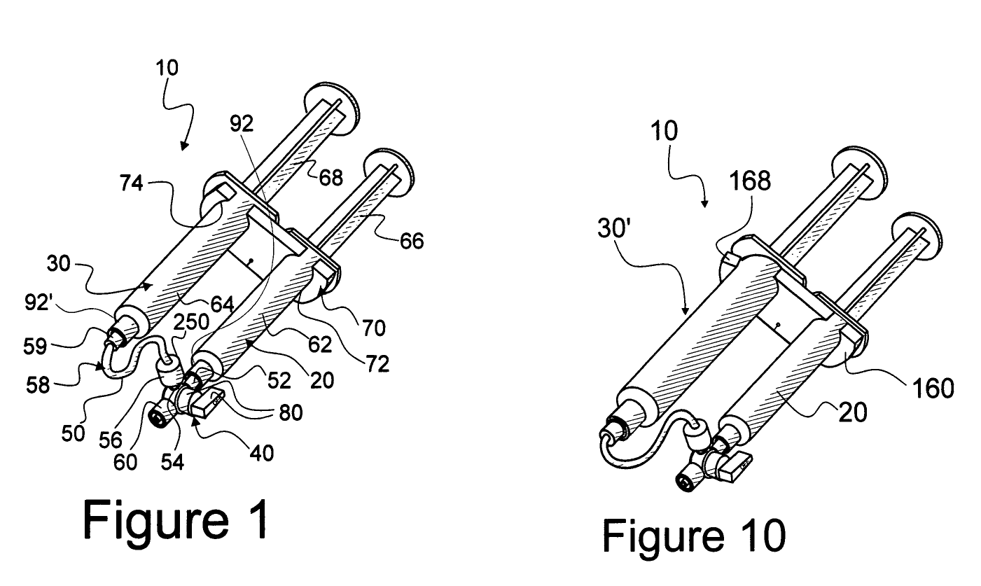

In this description, the term “proximal” indicates the segment of the device normally closest to the object of the sentence describing its position The term distal refers to a segment oppositely disposed. Reference is now made to the embodiments illustrated in FIGS. 1-38 wherein like numerals are used to designate like parts throughout For parts which are similar but not the same as parts originally specified with a given number, a prime of the original numbers is used.

While kits made according to the invention may be configured to provide assemblies for many medical procedures, such as those, for example, involved with injections of Adenosine, antibiotics and drugs for home-care, emergency and pediatrics, disclosure of an exemplary application in the area of hazardous drugs is herein selected to provide details of the instant invention while clearly demonstrating critically important safety and time and work-saving features. Reference is now made to FIG. 1 wherein a first convenience kit assembly 10 is seen to be readied for dispensing of fluids from a pair of syringes, numbered 20 and 30. Relative to a user, syringes 20 and 30 are distally interconnected through a stopcock 40 and microbore-tubing (short extension) set 50. Each syringe 20 and 30 may be a conventional commercially available medical syringe. One syringe, in particular syringe 30, may be a commercially available pre-filled flush syringe.

Stopcock 40 has three ports, a first port 52 being a female, preferably luer lock, connector which is securely affixed to syringe 20; a second port 54 also being a female, preferably luer lock, connector for connecting to a male connecting port 56 of tubing set 50. At an opposite end, tubing set 50 has a female, preferably luer lock, fitting 59 for secure attachment to syringe 30. Note that port 54 of stopcock 40 is disposed at right angles relative to port 52. Compliance and flexibility of tubing 58 of tubing set 50 permit syringe 30 to be aligned with syringe 20 for purposes disclosed in detail hereafter. A male, preferably luer lock, fitting 60 is exposed for attachment to a port, e.g. an injection port or a vial adapter, where through fluid is communicated.

Further, each syringe, numbers 20 and 30, has a barrel, generically numbered 62 and 64, respectively, and a plunger rod, also generically numbered 66 and 68, respectively. Note that plunger rods 66 and 68 are disposed well outside barrels 62 and 64 indicating both syringes 20 and 30 are filled to a predetermined level (of liquid).

Alignment of syringe 20 to syringe 30 is maintained and assured by a clip 70 having a pair of substantially circular, open slots 72 and 74, Slots 72 and 74 are shaped and formed to provide a releasible support for barrels 62 and 64, respectively. So configured, clip 70 provides a handle or grip whereby first and third fingers of a hand may be disposed outside a perimeter of barrels 62 and 64 with a middle finger of the same hand disposed between the barrels, thereby permitting the thumb of that hand to act upon either plunger rod as desired.

Fluid flow from assembly 10 is controlled by position of rotation of a core and handle 80 of stopcock 40. As seen in FIG. 1, fluid communication into and from syringe 20 is obstructed by the position of core and handle 80. As is explained in detail hereafter, rotation of core and handle 80 to a position obstructing outflow from syringe 30 opens outflow from syringe 20 to controllably permit selective dispensing of fluids from syringes 20 and 30 while keeping fluids within syringes 20 and 30 disparate. It is emphasized that use of a stopcock to control fluid flow is not the only way for such control; however, a stopcock as disclosed herein provides an efficacious way of dealing with requirements for keeping fluids separate in syringes 20 and 30.

### Stopcocks

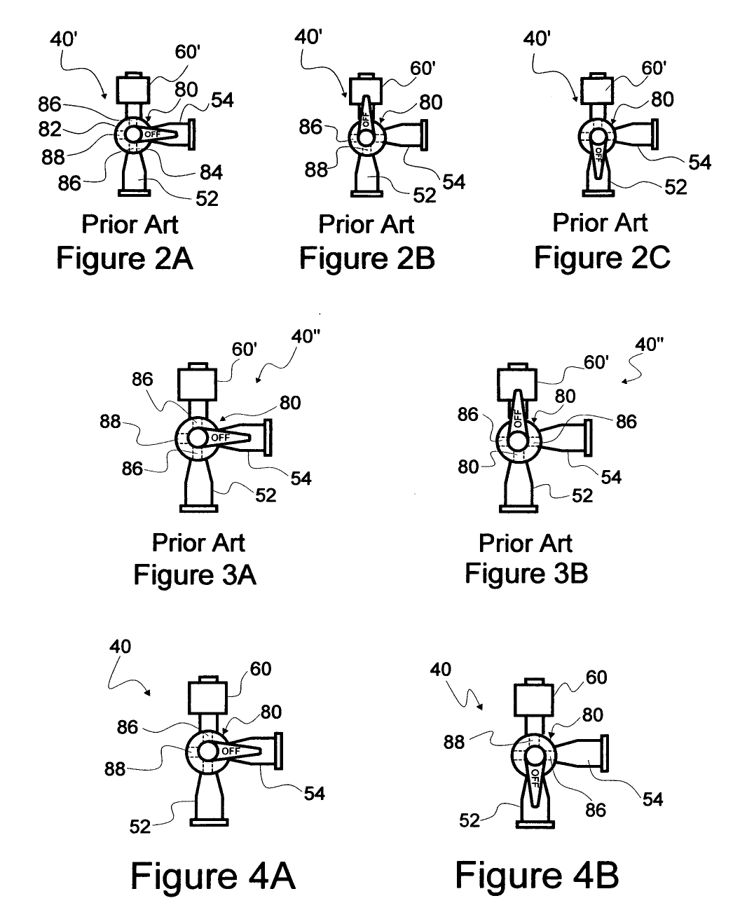

Generally, disposable stopcocks are well known and widely used in medical procedures. A three way stopcock 40′ which is commercially available is seen in FIGS. 2A, 2B and 2C. Stopcock 40′ has three ports 52, 54 and 60′ which, except for changes disclosed in detail hereafter, are substantially the same as stopcock 40 (see FIG. 1). As seen in FIG. 2A, within rotatable core 82 of core and handle 80, stopcock 40′ comprises a “T” shaped pathway 84 disposed to obstruct fluid flow through port 54 and permit fluid transmission between ports 52 and 60′. Note that pathway 84 can be considered to be comprised of two intersecting pathway segments, individually numbered 86 and 88. Pathway segment 86 is a through hole through core 82, while pathway segment 88 simply intersects pathway segment 86.

Rotating core and handle 80 to a stop associated with port 60′, closes port 60′ and permits fluid flow between ports 52 and 54 as seen in FIG. 2B. Rotating core and handle 80 to a stop associated with port 52 closes port 52 and permits fluid flow between ports 54 and 60′, as seen in FIG. 2C. Thus, stopcock 40′ is a three-way stopcock.

A stopcock 40″ seen in FIGS. 3A and 3B is also commonly found in contemporary commerce. Generally ports 52,54 and 60′ of stopcock 40″ are substantially the same as ports 52, 54 and 60′ of stopcocks 40 and 40′, except as disclosed in detail hereafter. Note that rotation of core and handle 80 to a stop associated with port 54 as seen in FIG. 3A, closes port 54 and permits fluid flow between ports 52 and 60′. However, rotation of core and handle 80 to a stop associated with port 60′, closes port 60′ and permits fluid flow between ports 52 and 54. In this manner, if port 60′ is an output connecting port and ports 52 and 54 are connected to syringes, the syringe connected to port 54 cannot communicate directly with port 60′. For this reason, a syringe connected to port 60′ is usually affixed thereto to provide dilution fluid to contents of a syringe affixed to port 54. Following such dilution, contents of the syringe affixed to port 52 are dispensed through port 60′.

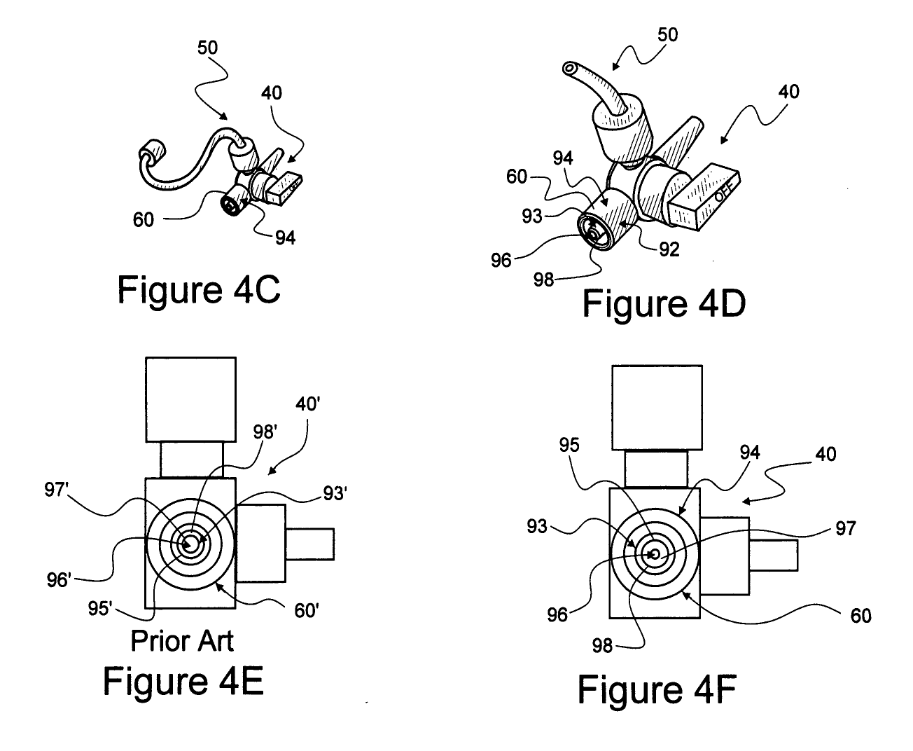

However, in an application where hazardous drugs are to be kept disparate from flushing fluids, it is important that there is no fluid communication between syringes containing such liquids. Therefore, as seen in FIGS. 4A and 4B, core and handle 80 of stopcock 40 rotation is stopped such that there is no simultaneous communication between ports 52 and 54 along pathway segments 86 and 88. Note, that when core and handle 80 is disposed at a stop associated with port 54, port 54 is closed (switching component state one). When core and handle 80 is disposed at a stop associated with port 52, port 52 is closed (switching component state two). Since stopcock 40 is a two way stopcock, no communication is permitted between ports 54 and 52. In this manner, fluid disposed within port 54 is kept disparate from fluid disposed within port 52.

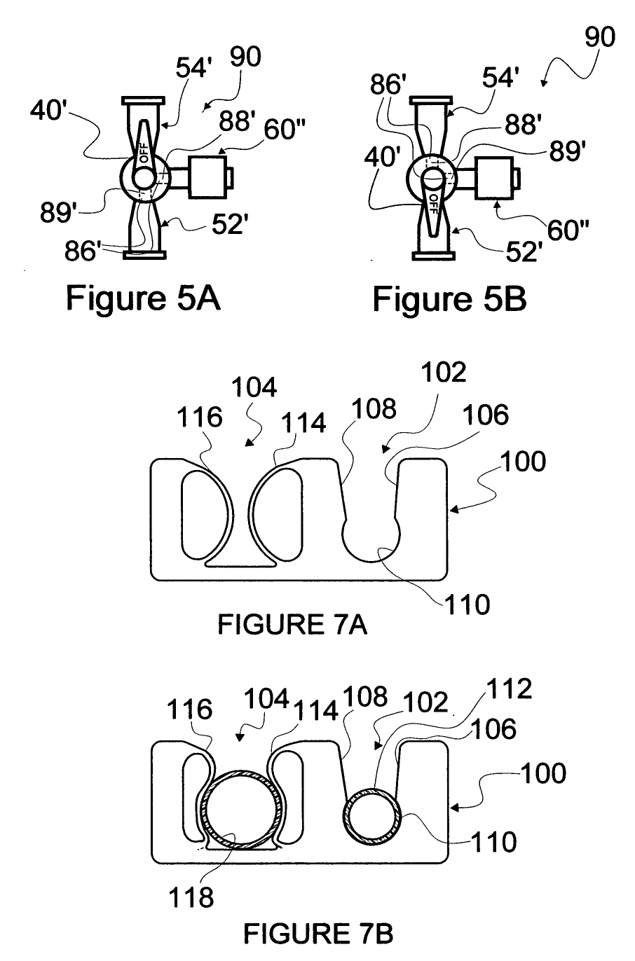

Another stopcock 90 seen in FIGS. 5A and 5B, like stopcock 40, also keeps fluids associated with a pair of syringe ports 52′ and 54′ disparate. Note in FIG. 5A that port 54′ is disposed in line with port 52′. However, a fluid pathway 86′ made up of two connected orthogonally disposed segments 88′ and 89′ permits fluid flow from only one side port 52′ or 54′ to a common output port 60″ at a time. Note in FIG. 5A that pathway 89′ leads from port 52′ to pathway 88′ and output port 60″ where core and handle 80 is at a stop associated with port 54′. Similarly, in FIG. 5B pathway 88′ leads from port 54′ to pathway 89′ and output port 60″ when core and handle 40′ is at a stop associated with port 52′. While port connections keep fluids of ports 52′ and 54′ disparate in the same manner fluids of ports 52 and 54 are disparate in stopcock 40 (see FIGS. 4A and 4B), dead space is decreased in stopcock 40′ (see FIGS. 5A and 5B) relative to dead space in stopcock 40 due to a pathway 89′ which is half the length of pathway 86.

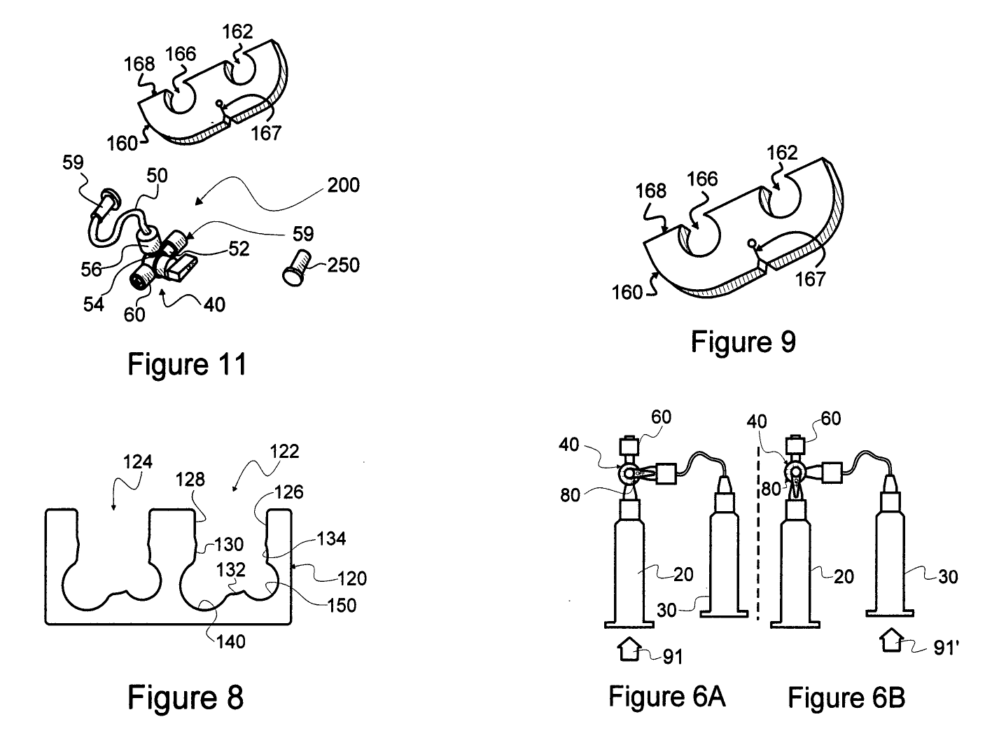

The need for a stopcock such as stopcock 40 (or 90) is exemplified by procedures for use as depicted in FIGS. 6A and 6B. In FIGS. 6A and 6B, arrows replace plunger rods showing direction of displacement of plunger rods. No arrow indicates no plunger rod movement. As a medical procedure associated with the instant invention involves, as a first step, delivering a dose from a syringe dedicated to providing a medication into an injection port. As a second step, immediately dispensing a flush solution into the injection port to flush both the injection port and the catheter, itself

Such is accomplished by simply rotating core and handle 80 to occlude the output pathway of syringe 30, as seen in FIG. 6A, and displacing the plunger rod of syringe 20 in direction of arrow 91. Once desired contents of syringe 20 are dispensed, core and handle 80 of stopcock 40 are displaced to occlude output of syringe 20, as seen in FIG. 6B, and displacement of the plunger rod of syringe 30, in direction of arrow 91′, provides flush solution to the injection port, IV set and catheter.

Commonly, needleless fittings are currently used as ports for IV sets affixed to patient catheters and contemporary vial adapters. These fittings have been designed to interface with male syringe luer fittings, such as luer fittings 92 and 92′, affixed to syringes 20 and 30, respectively, see FIG. 1. By standard specifications, male luer fittings have common outside diameters and frustoconical shapes. However, a review of inside diameters of male luer fittings reveals a marked difference between syringe male luer fittings and male luer fittings found on contemporary commercial stopcocks. Exemplary stopcock luer fittings are seen in FIGS. 4C-4F.

A stopcock 40 affixed to a tubing set 50 is seen in FIGS. 4C and 4D. Stopcock 40 and tubing set 50 are magnified in FIG. 4D for clarity of presentation with tubing set 50 truncated. As seen in FIG. 4D, a proximally disposed luer fitting 60 has a male luer part 93 surrounded by a luer lock 94. As stated supra, luer part 60 has an outside surface 98 having a standard luer diameter and frustoconical shape, and, to meet requirements of interfacing with needleless connectors, has a medially disposed through bore hole 96.

Stopcock 40 is further magnified in FIG. 4F for additional clarity of presentation. To aid in understanding a basic difference between stopcock 40 and PRIOR ART stopcocks, an example of a PRIOR ART stopcock 40′ is provided in FIG. 4E for comparison. Attention is drawn to male luer part 60 of stopcock 40 in FIG. 4F and a similar male luer part 60′ of stopcock 40′ in FIG. 4E. Note that stopcock 40 has a through bore hole 96 which is relatively smaller than a through bore hole 96′ of stopcock 40′ (see FIG. 4E).

Thus, associated proximal luer face 97, disposed between outside surface 98 and through bore hole 96 is larger in surface area than an outside surface 98′, disposed between outside surface 95′ and through bore hole 96′ of stopcock 40′. Generally, in the past, it is believed that through bore hole 96′ in stopcocks has been defined by draft specifications associated with injection molding. These draft specifications have resulted in the relatively larger size of bore hole 96′. It should be noted that such luer faces are circumferentially defined by outside surfaces having a smallest diameter of approximately 0.150 inches. Such is also true of stopcocks 40 and 40′.

However surface area of syringe luer faces are further defined by a through hole, similar to luer hole 96 of stopcock 40. Diameter of such a syringe through hole 96 is approximately 0.080 inches. Notably, diameter of an exemplary through hole 96′ for stopcock 40′ is approximately 0.120 inches. Note that a 0.120 inch diameter through hole yields a luer face width of about 0.015 inches while a 0.080 inch diameter yields a luer face width of about 0.035 inches. Such a difference in thickness of a luer face is a significant determinant in providing a reliable interface to a needleless connector which has been designed for use with syringe luer fitting dimensions. It is for this reason that stopcock 40 has a significantly smaller through hole diameter than stopcock 40′. Such a decreased size in luer diameter may be achieved by a change in mold design or by affixing a tube having a desired through hole diameter into a larger through hole, such as through bore hole 96′. For purposes of reference, such a stopcock, having a bore hole and luer face thickness similar in dimension and function to a syringe luer connection, is further referenced herein as a needleless compatible connector.

### Clips

Referring once more to FIG. 1, please note that syringe 20 and syringe 30 are aligned, one relative to the other, and held in alignment by clip 70. Clip 70 provides a releasible attachment for each syringe to improve facility of operation of two syringes held in a single hand. Note that a first and third finger may be placed about syringes 20 and 30 while a middle finger may be placed between the two syringes in such a manner that the thumb of the hand can be used to displace each syringe rod, 66 and 68. It is important that clip 70 holds each syringe securely, but releasibly, such that either syringe may be removed from clip 70 for purposes which require a separated syringe, such as placing a syringe in a syringe pump.

Clips for assembly 10 may be made in many forms within the scope of the instant invention. Basic criteria for such clips are that the clip must provide sufficient stability for assembly 10 that two syringes may be facilely employed in a single hand and the syringe attachment must be secure, but releasible. Another optional requirement is that the clip be usable for a predetermined range of syringe barrel sizes.

A syringe clip 100, made according to the instant invention, is seen in FIGS. 7A and 7B. Syringe clip 100 has a pair of slots 102 and 104 into which syringes may be displaced. As seen in FIG. 7A slot 102 comprises a pair of sides 106 and 108 which converge toward an open circular slot 110 which is sized and shaped to conform to a single predetermined syringe barrel size. Note, in FIG. 7B that a syringe barrel 112 (seen in cross section), is disposed in slot 102.

However, it is preferred that a clip be useful for more than one syringe barrel size. For this reason, slot 104 comprises a pair of compliant ribs 114 and 116 which forgivingly separate when a syringe barrel is displaced there into (see a cross section of a syringe barrel 118 disposed in slot 104. Ribs 114 and 116 must exert sufficient force against barrel 118 to retain barrel 118 in slot 104 once so disposed.

Preferably, clip 100 should be sufficiently thick to hold each inserted syringe barrel in position throughout a predetermined medical procedure associated with assembly 10. Clips like clip 100 may be injection molded using polypropylene.

A clip which is specifically designed to hold syringe barrels of a variety of sizes is seen in FIG. 8. As seen in FIG. 8, a clip 120 comprises two identical slots 122 and 124. As slots 122 and 124 are identical, only characteristics of slot 122 will be disclosed in detail. Slot 122 has a pair of converging sides 126 and 128 and a pattern which is sized and shaped to grasp a large syringe barrel (not shown) within edges 130, 132 and 134. Offset from slot 122 is a smaller circular slot 140 which is sized and shaped to grasp a smaller syringe barrel (also not shown). On an opposite side of slot 122 is yet another still smaller circular slot 150 sized and shaped to grasp a still smaller syringe (also not shown). In this manner, a single clip 120 may be used to hold one of three different sized syringe barrels within each slot 122 and 124. Similar to clip 100, clip 120 may be injection molded from polypropylene or other suitably stable plastic material.

A preferred clip 160 is seen in FIG. 9. Clip 160 comprises a pair of circular slots, numbered 162 and 166, which open to permit insertion (and retrieval) of a syringe barrel. The circular slots each have a diameter which is smaller than the smallest syringe barrel specified for use in assembly 10. Further, clip 160 has a centrally disposed slit and hole 167 sized and shaped to permit clip 160 to be facilely attached and suspended from tubing available at the site of use.

Clip 160 is preferably made of a substantially rigid closed cell foam material. As such clip 160 may be made by stamping out of a sheet of material. While clip 160 may be made in various thicknesses (e.g. from 0.25 to 0.5 inches), a thickness of 0.5 inches is presently preferred. Closed cell foam, from which clip 160 is made, is particularly compatible for use as a barrel holder for assembly 10. Such foam permits a tight grasp of an inserted barrel and yields when a much larger barrel is inserted to provide a stabilizing clasp upon the larger syringe barrel.

An example of the manner in which clip 160 yields to a larger syringe is seen in FIG. 10 wherein an assembly 10 comprises a syringe 30′ which is substantially larger in diameter than syringe 30 as seen in FIG. 1. Note in FIG. 10 that an outside arm 168 of clip 160 is displaced from an original position as seen in FIG. 9. While insertion of larger syringe 30′ causes displacement of arm 168, clip 160 still acts as an adequate stabilizing clasp about syringe 30′.

### Kit Packaging

Generally, kit components, to be sterilize, are packaged into a covered, sealed pouch, which is sterilized by a predetermined method of sterilization (such as gamma radiation, ethylene oxide, etc.). One of the primary objects of the first kit is to decrease numbers of makes and breaks after sterilization to as few as possible. For this purpose, where possible, kit parts which are joined for use in assembly 10 are affixed one to another prior to being sterilized. It is important that these parts remain securely affixed one to another through all phases of kit use.

For this reason, it is recommended that these parts be unitized parts, becoming even as a single unitized part 200 (i.e. be adhesively interconnected where possible), as seen in FIG. 11. Where such is not possible the parts should be tightly mechanically secured. In part 200, port 54 of stopcock 40 is affixed to a male fitting 56 of a short extension set 50. A female fitting 59 and a male fitting 60 are left open for purposes which are disclosed in detail hereafter. As a cap 250 may be later used as a sterility protecting cover after a syringe 20 filing procedure, cap 250 is included with the other sterilized parts. Also included, for convenience, is a clip 160.

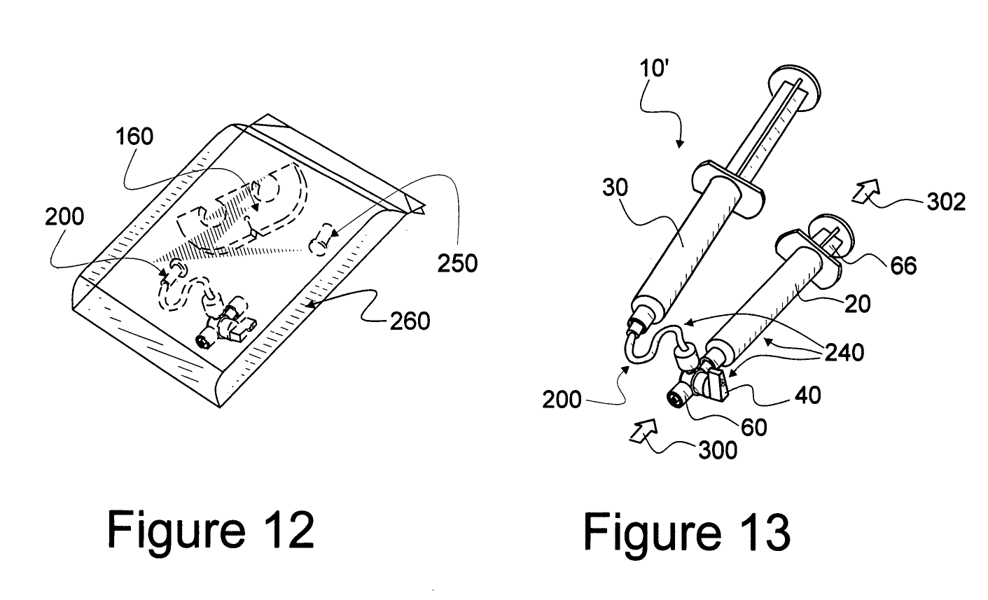

A preferred mode of packaging a kit for part 200, cap 250 and clip 160 is seen in FIG. 12. Note that the kit parts are disposed and sealed within a sterilizable peel pouch or wrap 260 wherein contained parts are sterilize by a preselected process (e.g. gamma radiation).

Note that dose syringe 20 and flush syringe 30 are not included in items sterilized in peel pouch or wrap 260. Generally, both syringes are readily available at a using institution and a kit having a particular syringe may not match syringes selected for use by that institution.

### Alternative to Stopcock

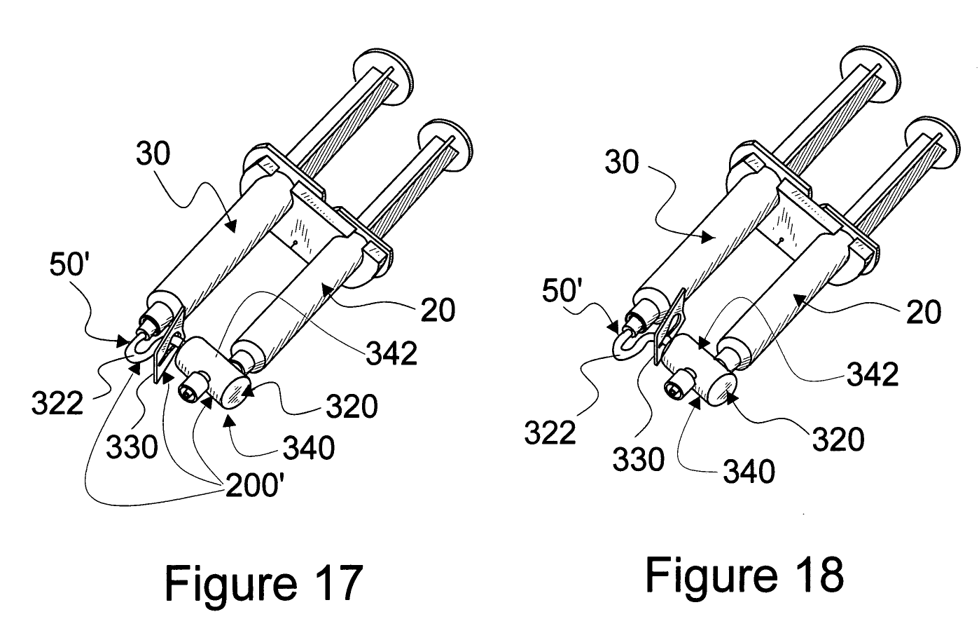

While use of a stopcock, such as stopcock 40, is in accord with the first kit, an alternative, which requires no external manual switching is provided by a pressure actuated fluid switching apparatus 320, seen in FIGS. 17-21. As seen in FIG. 17, apparatus 320 is affixed to a dose syringe 20 and a flush syringe 30 in the same manner that dose and flush syringes are affixed to a stopcock 40. Interposed between flush syringe 30 and apparatus 320 is a length of microbore-tubing 322 which is part of a short extension set 50′ which is similar in form and function to set 50, disclosed supra. In the case of apparatus 320, each syringe 20 and 30 may be a conventional commercially available medical syringe. A tubing clamp 330 is affixed about tubing 322 on short extension set 50′ for purposes which are disclosed in detail hereafter. Similar to part 200 (see FIG. 11), apparatus 320, set 50′ and clamp 330 are assembled and, where possible, adhesively affixed to form a unitized part 200′. As such part 200′ may be used in place of part 200.

As better seen in FIG. 19, similar to stopcock 40, switching apparatus has three ports, a first port 52′ being a female, preferably luer lock, connector which is securely affixed to syringe 20. Port 52′ is an integral part of a housing 340 which has two other integrally formed ports, a second port 56′, which is similar in function to port 56, and a third port 60 which is identical in form and function to earlier disclosed port 60 (see FIG. 1).

Housing 340 further comprises a hollow cylindrical core 342 which is dead-ended at a face 344 which is disposed to permit communication between core 342 and port 52′ and to be open at the other end 346. A closing cap 350 is sized and formed to provide a stopper at end 346. Cap 350 has a medially disposed through hole 352 into which tubing 322 is securely (preferably adhesively) affixed. Port 60, seen without a luer lock fitting for clarity of presentation, provides a communicating fluid transfer port for ports 52′ and 56′.

Disposed within core 342 is a displaceable plug 360 having a first facing end 362 and a second facing end 364. Plug 360 is sized and shaped to keep fluids which communicate with first end 362 and second end 354 disparate. Also disposed within core 342 is a spring 370.

Housing 340 and cap 350 may be injection molded using stable and substantially medically inert plastic materials such as polypropylene. Plug 360 may be made from materials which are used for syringe plungers, such as butyl rubber. Spring 370 may be made from any material which is compressible and has stored force return memory, such as stainless steel.

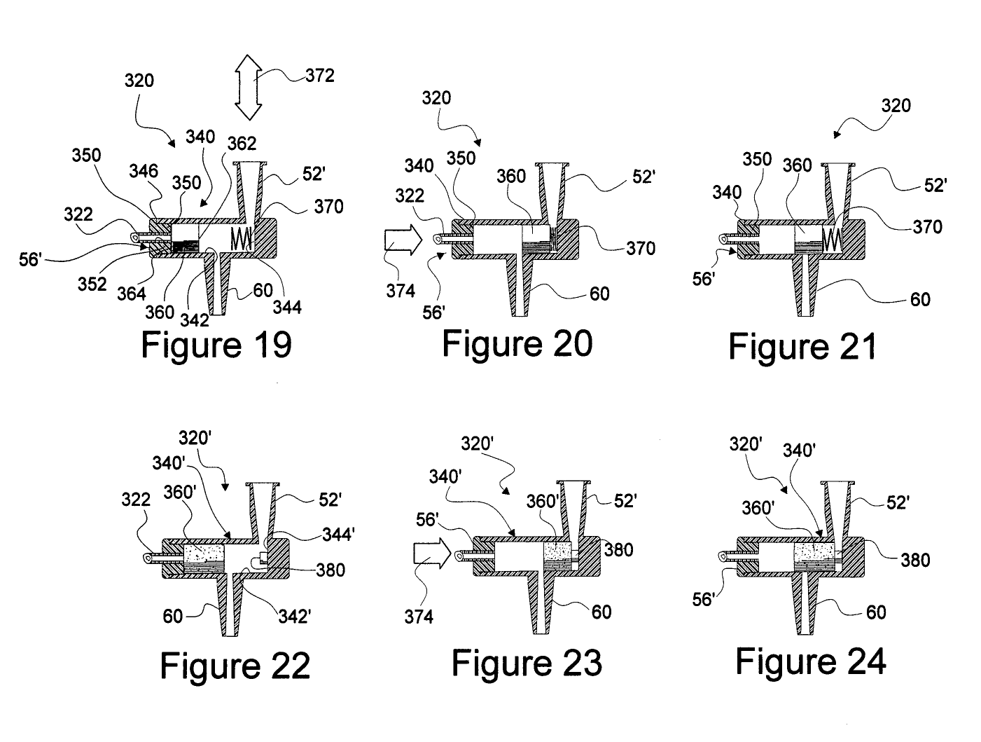

As seen in FIG. 19, plug 360 disposition closes port 56′ to flow from a connected syringe 30. In this state (state 1), fluid may be displaced in two directions as indicated by double ended arrow 372. Should a negative pressure be applied by syringe 20 to draw fluids through port 60 be sufficient to displace plug 360 to thereby block flow from port 60 to port 52′, such displacement of plug 360 can be restrained by clamping tube 322 with clamp 330, as seen in FIG. 18. Then, when pressure communicated through port 52′ is relieved by disuse of an attached syringe 20 and pressure is applied via a syringe 30 in direction of arrow 374 (see FIG. 20), plug 360 is displaced to permit fluid communication between port 56′ and port 60 (state 2). Note that spring 370 is compressed to permit such communication.

When force on plunger of syringe 30 is released, energy stored in spring 370 displaces plug 360 to block fluid flow between port 60 and port 56′, as seen in FIG. 21. As may be noted, such action of spring 370 upon plug 360 blocks reflux flow from port 60 to port 56′, thereby keeping liquid in syringe 30 uncontaminated by fluids being transported through port 60. In this manner, fluid may be bi-directionally displaced using syringe 20, but only dispensed from syringe 30.

Another pressure actuated fluid switching apparatus 320′ is seen in FIGS. 22-24. With a single exception housing 340′ of apparatus 320′ may be identical to housing 340 of apparatus 320 (see FIG. 19). Note that a rear face 344′ within core 342′ is interrupted with a medially disposed stop 380. Rather than an incompressible plug, such as plug 360 (see FIG. 19), apparatus 320′ has a plug 360′ made from compressible material, as seen in FIG. 22.

Plug 360′ is disposed in state 1 in FIG. 22. In state 1, as disclosed supra for other embodiments, fluid communication is permitted between port 52′ and port 60. As shown in FIG. 19, fluid may flow bidirectionally between ports 52′ and 60. Application of pressure through tubing 322 (from a syringe 30) displaces plug 360′ to become displaced against stop 380. Further pressure causes plug 360′ to compress as seen in FIG. 23 to open fluid communication between port 56′ and 60 (state 2). Note that, when pressure from syringe 30 is relieved, plug 360′ expands to block flow between ports 56′ and port 60, as seen in FIG. 24. In this manner, reflux flow is blocked between port 60 and port 56′ to assure that fluid in syringe 30 cannot be contaminated by reflux flow through port 60. A candidate material which may be used for plug 360 is a medical grade SANTOPRENE® TPZ 18-55, by EXXON Mobil.

### Methods of Kit 1 Preparation and Use

Reference is now made to FIG. 13 wherein an assembly 10′ kit 1 parts, which is assembly 10 without clip 70, is seen. Note that assembly 10′ (a part of assembly 10) is constructed by attaching an empty dose injection syringe 20 and a pre-filled flush syringe 30 to a unitized part 200 (see FIG. 13) to make a completed assembly. To reduce likelihood of contamination, such attachments should be performed in a clean, controlled environment, such as within a safe area of a laminar flow hood.

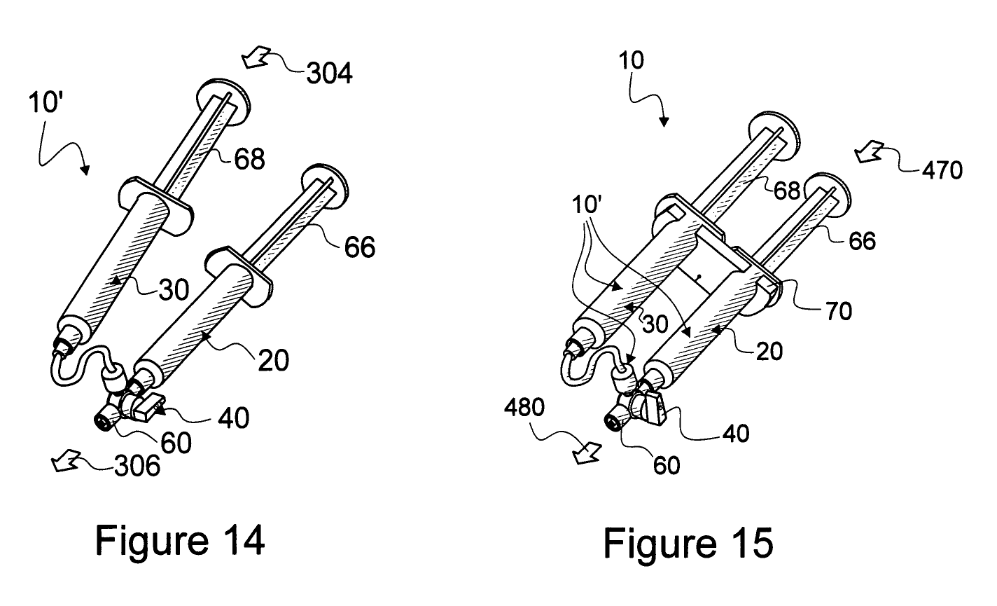

With stopcock 40 disposed for filling syringe 20, as seen in FIG. 13, male fitting 60 of stopcock 40 is affixed to a source of drug (e.g. a vial adapter having a needleless connector) to be transferred to syringe 20 according to institutional protocol. As is well understood by clinicians trained in use of syringes, medication or drug is drawn into syringe 20 in direction of arrow 300 by retracting plunger rod 66 in direction of arrow 302. Once syringe 20 is filled, and primed, a predetermined amount of flush solution is dispensed from pre-filled flush syringe 30 as seen in FIG. 14. To accomplish this, stopcock 40 is disposed to permit fluid flow from syringe 30 through fitting 60. Plunger rod 68 is displaced in direction of arrow 304 to dispense flush solution outward from fitting 60 in direction of arrow 306. It is recommended that, for 10 ml flush syringes, one to two milliliters of flush solution be dispensed through flush fitting 60, though volumes may vary dependent upon character of drug in syringe 20. Note that by flushing fitting 60 hazardous drug resident at fitting 60 is displaced by flush solution.

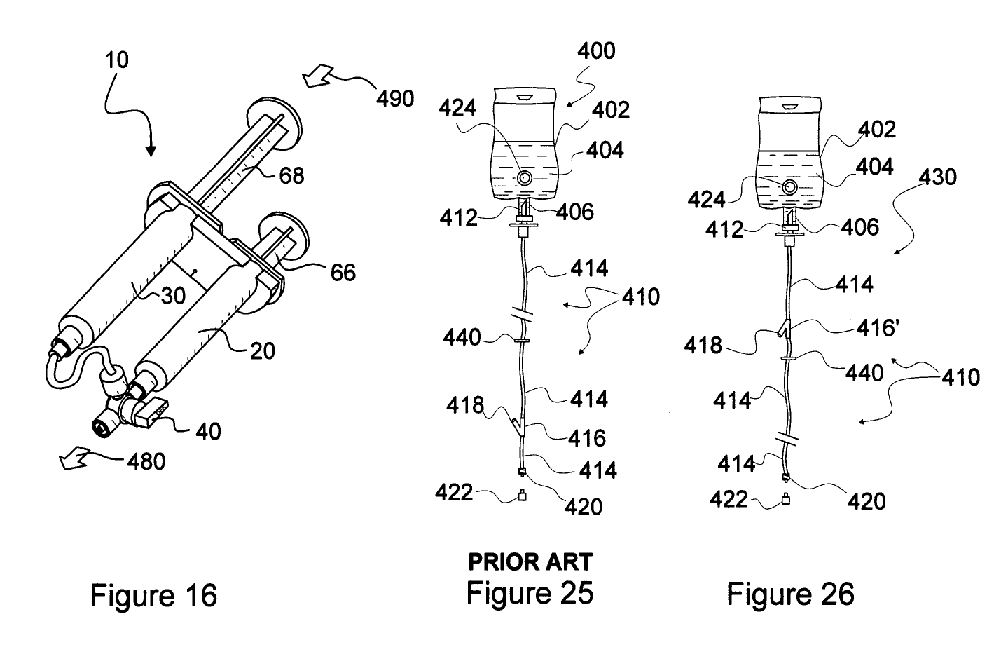

Once syringe 20 is filled and fitting 60 is flushed, stopcock 40 should remain in the open flush pathway state. Fitting 60 should be capped (preferably with provided cap 250 (see FIG. 11)). At any desired time, clip 70 may be affixed thereto (as seen in FIGS. 1 and 16) to form assembly 10. Then, following institutional protocol, a prepared system 10 is delivered to a site of use.

A site where a drug is dispensed from syringe 20 may be varied. Examples of such sites are provided hereafter:

### Dispensing in Pharmacy

A first exemplary site for use of parts from Kit 1 is in pharmacy, likely where a system 10 is prepared. In such a case, medication may commonly be dispensed into an IV container through some kind of injector site. A pathway for injecting might include a secondary spike injection site, use of a female/female adaptor for drug delivery through a distal tip of a secondary IV piggyback set, or a side injection port or a pathway through an associated IV set.

An exemplary PRIOR ART IV solution bag/IV set combination 400 is seen in FIG. 25. Combination 400 generally comprises an IV container 402 filled with IV solution 404, a spike insertion port 406 and an injection set 410 having a superiorly disposed spike 412 (introduced into port 406), an elongated length of medical tubing (generally numbered 414), an inferiorly disposed Y-site 416 (usually fitted with a needleless connector 418) and a needleless connector compatible fitting 420 for being ultimately affixed to a patient catheter or an IV extension set. A tip cap 422 is also provided to close IV set 410 for transport.

Note that Y-site 416 is disposed for inferiorly directed injection into tubing 414, likely at a patient site. Note also that IV bag 402 has an injection port 424 where through a medication may be dispensed by needle insertion. As ports, like port 424, may leak, such are not considered by inventors to be appropriate interfaces for hazardous drugs.

To provide a safer interface for dispensing hazardous drugs into an IV bag, such as bag 402, an IV solution container/IV set combination 430, made according to the present invention is seen in FIG. 26. Combination 430, as an example, may comprise an IV bag 402 filled with IV solution 404, a spike insertion port 406 and an injection set 410 having a superiorly disposed spike 412 (introduced into port 406), an elongated length of medical tubing (generally numbered 414), a Y-site 416′ (superiorly disposed relative to Y-site 416 and fitted with a needleless connector 418) and a needleless connector compatible fitting 420 for being ultimately affixed to a patient catheter. A tip cap 422 is also provided to close IV set 410 for transport.

Note that Y-site 416′ is disposed for superiorly directed injection into tubing. Note also that IV bag 402 injection port 424 is not needed as Y-site 416′ may be safely and efficaciously used for dispensing medication into solution 404. Note: Before dispensing medication into bag 402, assembly 410 should be primed with solution from the IV container. Then, with assembly 10 (see FIG. 1), affixed to Y-site 416′ through needleless connector 418 a medication/flush cycle may begin. A slide clamp 440 inferiorly disposed, relative to Y-site 416′, is oriented about tubing 414 to occlude tubing 414.

In the case of assembly 10, stopcock 40 is adjusted to provide a pathway from syringe 20 through luer connector 60 and there through Y-site 416′ and into bag 402 and solution 404. Once a desired volume of medication is dispensed, stopcock 40 is adjusted to provide a pathway from syringe 30 through luer connector 60 and needleless connector 418 and into bag 402 to permit flushing of luer connector 60, needleless connector 418, Y-site 416′, tubing 414 superior to Y-site 416′ and associated spike 412. Once flushing is completed, assembly 10 may be removed with safety and slide clamp 440 adjusted to permit flow through tubing 414. For safety, assembly 10 should be disposed of following institutional protocol.

### At Patient Dispensing

Note that, when, for example, assembly 10 is displaced for use at a medication delivery site, a clinician may perform the dispensing operation single handed, dispensing at will from either of the two syringes, 20 and 30. For catheter related dispensing, such as through a Y-injection site or an IV set and through a catheter, fitting 60 is securely, but releasibly affixed to a receiving catheter or other receptacle fitting (at least for hazardous drugs, the fitting should be a needleless connector). Stopcock 40 is set to provide an open pathway from syringe 20 to fitting 60 (see FIG. 15). Plunger rod 66 is displaced in direction of arrow 470 to dispense medicament through fitting 60 in direction of arrow 480 for its designated purpose, as seen in FIG. 15. Note that by grasping assembly 10 about syringes 20 and 30 with the index and third fingers and placing the middle finger of a hand between syringes 20 and 30, the thumb of the hand can facilely displace plunger rods 66 and 68.

Once a desired amount of fluid of syringe 20 is displaced therefrom, stopcock 40 is displaced to obstruct flow of fluid from syringe 20 and open the fluid flow pathway from syringe 30. Generally, sufficient fluid is dispensed from syringe 30 by displacing plunger rod 66 in direction of arrow 490 to flush fitting 60, an associated IV connector and a catheter or other communicating fluid line, as seen in FIG. 16. For a single use application of assembly 10, plunger rods 66 and 68 are fully displaced, spent assembly 10 is delivered to a disposable receptacle according to institutional protocol.

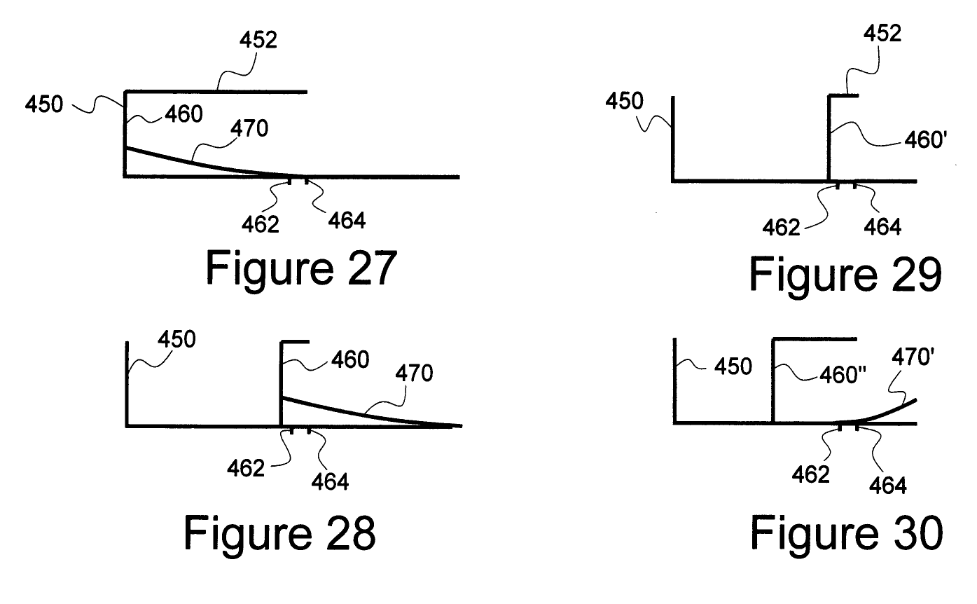

In some institutions, it is a practice to attempt to flush a catheter connector (usually needleless) by drawing flush, into the syringe from which medication was dispensed, from a saline drip line following medication delivery. Applicants feel a necessity to stipulate a concern relative to such a practice. As an example, such a practice may yield a distribution of medication following such flushing as indicated in FIGS. 27 and 28.

In graphs of FIGS. 27-30, the “Y” or vertical axis 450 of each graph represents a measure of drug concentration. The “X” or horizontal axis of each graph a measure of relative distance between points enumerated on the “X” axis (effectively plunger rod displacement). A relative disposition of an exposed face of a syringe plunger which is used to displace fluid from a syringe is designated by vertical line 460. A small vertical line 462 designates position of a needleless connector interface and a second small vertical line 464 designates position of a point where an attached section of a “Y” connector communicates with an associated IV set. A downwardly sloping line 470 indicates a concentration gradient away from a syringe plunger tip face 460.

As seen in FIG. 27, an original medication concentration 452 is contained in and delivered from, in the more general case, a medication syringe. An attempt to draw in saline solution from a saline drip line for a flushing purpose results in some mixing and a concentration gradient which is plotted for example as gradient 470 as seen in FIG. 27. Note that concentration of gradient 470 is highest at plunger face 460 where actual concentration is dependent upon mixing between original medication resident in dead space (including the attached section of the “Y” connector. When plunger face 460 is displaced to flush past points 462 and 464, concentration gradient 470 is effectively displaced to provide the highest level of concentration in the region of points 462 and 464. Because, point 462 represents a break point when the medication syringe is detached from the “Y” site, some concern is believed to be in order.

On another hand, if assembly 10 (see FIG. 1) is used for flushing, with a pre-filled flush syringe providing flush solution external to a “Y” site, concentration at the end of dispensing yields an original concentration 452 at a driving plunger face (e.g. face 460′ in FIG. 29). Interestingly, when a stopcock is switched, such as stopcock 40 of assembly 10, medicine concentration within a pre-flush syringe is inherently free of medication (having a zero medication concentration). Therefore, as a driving plunger face (e.g. face 460″ of a pre-filled syringe 30) is displaced to dispense flush from syringe 30, concentration gradient is generally of the form of curve 470′ of FIG. 30. For this reason, it is intuitive that flushing is more efficacious when using a flush syringe than when using a medicine delivery syringe to draw and redispense a saline/medicine mix in an attempt to clear a needleless connector and associated “Y” site.

### Alternative Pharmacy Dispensing

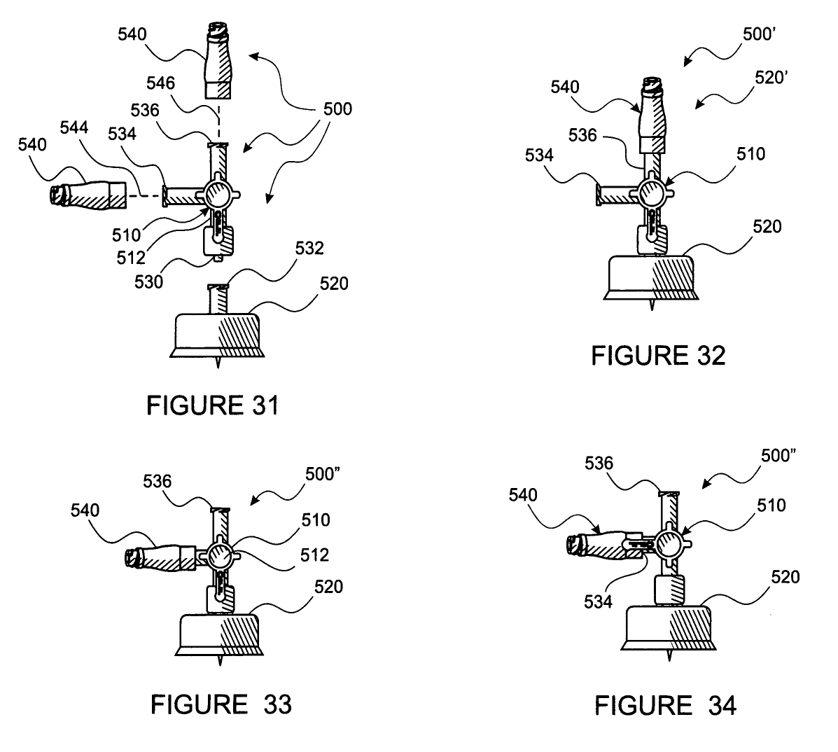

In applications where Kit 1 assemblies cannot be used efficiently (to reduce makes and breaks) when transferring fluid from vials, use of Kit 2, referenced hereafter as kit 500, provides a unique and expeditious opportunity. Like Kit 1, kit 500 is fabricated from components which are generally familiar to clinicians. As seen in FIG. 31, kit 500 is comprised of a stopcock 510 (with an associated core and handle 512 for fluid pathway selection) or other switchable pathway selecting device and an associated vial adapter 520. Preferably stopcock 510 and vial adapter 520 are adhesively attached at a male luer connection 530 of stopcock 510 and a female luer connection 532 of vial adapter 520. Noting that stopcock 510 has two female luer connections, numbered 534 and 536, a needleless connector 540 may be connected to either female connection 534 or female connection 536 as indicated by dashed lines 544 and 546, respectively.

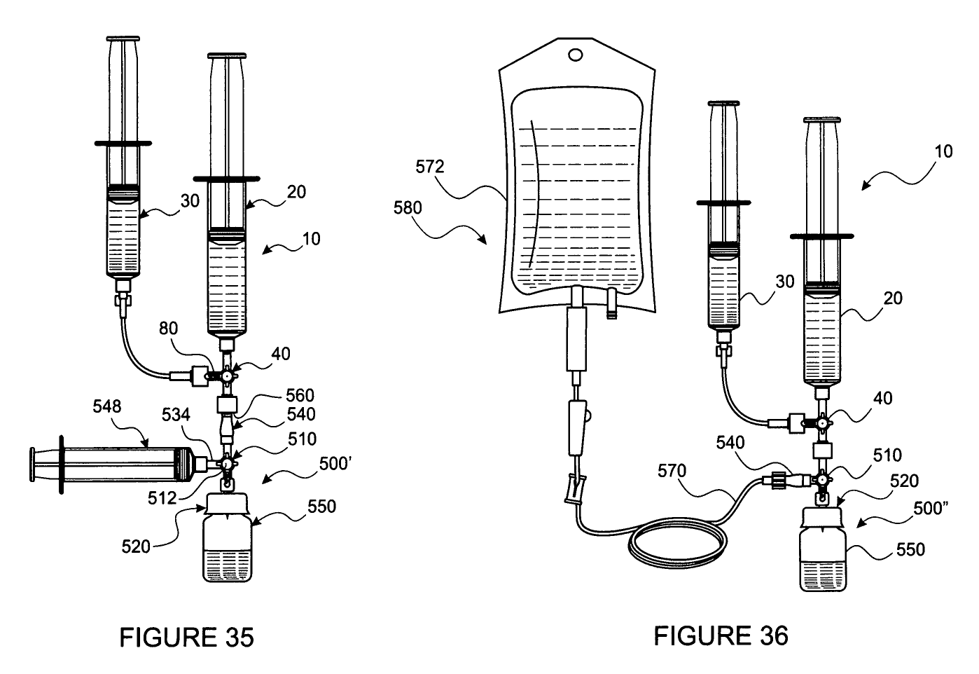

An embodiment of an assembled kit 500, referenced as kit 500′, is seen in FIG. 32. Kit 500′ is configured with needleless connector affixed to female connection 536. In this embodiment (for preparing assembly 10 for bolus delivery at a patient site), kit 500′ is affixed to an assembly 10 via needleless connector 540 as seen in FIG. 35. In addition, a syringe 548 destined to receive waste fluids is affixed to female connection 534. A medicant containing vial 550 is affixed to vial adapter 520 following institutional protocol.

A procedure for use of kit 500′ may be as follows:
1. If contents of vial 550 are lyophilized and require reconstitution a pathway for fluid delivery from syringe 30 to vial 550 is opened vial stopcocks 40 and 510 and a predetermined volume of reconstitution liquid is dispensed into vial 550. Note that before any liquid is delivered into vial 550 it is advisable to withdraw sufficient gas from vial 550 into syringe 20 and therefrom into waste syringe 548 to establish a negative pressure in vial 500 so that fluid will not inadvertently escape from vial 550 during this procedure.
2. Stopcock 40 is then adjusted to open a pathway from vial 550 into syringe 20 and a predetermined volume of medicant is drawn into syringe 20. Stopcock 510 is then adjusted to provide a pathway from syringe 20 into syringe 548 so that syringe 20 may be primed.
3. Stopcock 40 is next adjusted to open a pathway from syringe 30 to syringe 548 and a predetermined volume of flush solution is delivered to syringe 548 from syringe 30. Note that delivery of flush solution to syringe 548 flushes a connection between stopcock 40 and needless connector 540 permitting assembly 10 to be separated from needleless connector without presenting concentrated medicant at the connection site 560.
4. Assembly 10 is then separated from assembly 500′ and appropriately prepared for delivery to a bolus delivery site.
   If an additional dose of medicant is desired to be accessed from vial 550, another assembly 10 is affixed to the same assembly 500′ as seen in FIG. 35 and steps 2-4 are repeated.

Another embodiment of an assembled kit 500, referenced as kit 500″ , is seen in FIGS. 33 and 34. Kit 500″ is configured with needleless connector affixed to female connection 534. In this embodiment, kit 500″ is affixed to an assembly 10 via female luer connection 536 as seen in FIG. 36. In addition, an extension set 570 and associated IV container (bag 572) which forms combination 580 to be delivered to a site of use. A medicant containing vial 550 is affixed to vial adapter 520 following institutional protocol. Note that, in this case, it is combination 580 which is delivered to a site of use rather than a pre-filled assembly 10 as is the case for use of assembly 500′.

A procedure for use of kit 500′ may be as follows:
1 If contents of vial 550 are lyophilized and require reconstitution a pathway for fluid delivery from syringe 30 to vial 550 is opened via stopcocks 40 and 510 and a predetermined volume of liquid is dispensed into vial 550. Before any liquid is delivered into vial 550 it is advisable to withdraw sufficient gas from vial 550 into syringe 20 to establish a negative pressure in vial 500 so that fluid will not inadvertently escape from vial 550 during this procedure.
2. Stopcock 40 is then adjusted to open a pathway from vial 550 into syringe 20 and a predetermined volume of medicant is drawn into syringe 20. Stopcock 510 is then adjusted to provide a pathway from syringe 20 extension set 570 so that medicant may be delivered to combination 580.
3. Stopcock 40 is next adjusted to open a pathway from syringe 30 to extension set 570 and a predetermined volume of flush solution is delivered to combination 580. Note that delivery of flush solution to combination 580 performs two critical functions. First, medicant is fully transferred into bag 572 for appropriate dilution. Second, a connection between needleless connector 540 and extension set 570 is flushed. Thus, combination 580 and assembly 500″ may be separated without concentrated medicant being present at the site of disconnection.
4. Combination 580 is then separated from assembly 500″ for delivery to a site of use.
   If an additional dose of medicant is desired to be accessed from vial 550, another combination 580 is affixed to the same assembly 500″ as seen in FIG. 36 and steps 2-4 are repeated.

What is claimed and desired to be secured by Letters Patent is:

1. A method for transferring medicants from a vial through a contiguous closed system with safety to a portion of the system which is thereafter separated and delivered to a site of use, said method comprising the steps of:
(a) providing a first convenience kit for assembling an apparatus for measuring, filling and dispensing medication and flush solutions comprising:
first fluid switching apparatus comprising:
a first female luer fitting (fitting I) for connecting to a medication measuring and delivery syringe, a second female luer fitting (fitting II) for connecting to a pre-filled flush syringe, and a third male luer fitting (fitting III) through which medication is accessed, measured and dispensed;
said first fluid switching apparatus further comprising three intersecting pathways and an associated first displace-able switching component disposed for selectively obstructing and permitting fluid flow through each of said intersecting pathways, a first pathway being disposed between said fitting I and said first displace-able switching component, said second pathway being disposed between said fitting I and said first displace-able switching component and said third pathway being disposed between said fitting III and said first displaceable switching component;
said first displaceable switching component comprising structure which, when displaced to a first state, selectively provides a fluid communication pathway only from said first pathway to said third pathway and when displaced to a second selected state provides a fluid communication pathway only between said second pathway and said third pathway, but under no conditions, in any state, provides a fluid communication pathway between said first and second pathways; and
said fitting III comprising structure which provides the pathway through which fluid is externally communicated; and
(b) providing a second convenience kit for assembling an apparatus for measuring, filling and dispensing medication and flush solutions comprising:
a second fluid switching apparatus comprising:
a first female fitting (fitting IV), a second female fitting (fitting V), and a third male fitting (fitting VI);
said second fluid switching apparatus also further comprising three intersecting pathways and an associated second displace-able switching component disposed for selectively obstructing and permitting fluid flow through each of said intersecting pathways of the second fluid switching apparatus, a first of such pathways of the second fluid switching apparatus being disposed between fitting IV and said second displace-able switching component, said second pathway of the second fluid switching apparatus being disposed between fitting V and said second displace-able switching component and said third pathway of the second fluid switching apparatus being disposed between said fitting VI and said second displaceable switching component;
said second displaceable switching component comprising structure which, when displaced to a first state, selectively provides a fluid communication pathway only between said first and said third pathways of the second desplaceable switching component and when displaced to a second selected state provides fluid communication only between said first and second pathways, but under no condition, in any state, provides a fluid communication pathway between said second and third pathways;
a vial adapter securely affixed to fitting VI; and
a needleless connector.

2. A method for transferring medicants according to claim 1 comprising a step (c) of providing fitting III with structure consistent with geometry required for connecting to a needleless connector.

3. The method for transferring medicants according to claim 3 comprising further assembly steps of:
(d) affixing said needleless connector to between fitting III and fitting IV, thereby providing a detachable connection between the needleless connector and fitting III;
(e) acquiring and affixing a residue container to fitting V;
(f) acquiring and affixing a medical syringe to fitting I;
(g) acquiring and affixing a pre-filled flush syringe to fitting II; and
(h) acquiring and securely affixing a vial containing a medicant to be transferred to fitting VI thereby providing a closed system comprising combined contents of said first and second kits.

4. The method for transferring medicants according to claim 3, if the medicant in the vial requires added liquid for reconstitution of lyophilized material, comprising further steps of:
(i) displacing the switching component of said first fluid switching apparatus to permit fluid communication between fittings I and III;
(j) displacing the switching component of said second fluid switching apparatus such to permit fluid communication between fittings IV and VI;
(k) drawing a predetermined volume of gas from the vial into the medication syringe to assure a negative transfer pressure;
(l) displacing the switching component of said first fluid switching apparatus such to permit communication between fittings II and III;
(m) dispensing a predetermined volume of liquid from the pre-filled flush syringe into the vial.

5. The method for transferring medicants according to claim 4 comprising further steps of:
(n) displacing the switching component of said first fluid switching apparatus such to permit communication between fittings I and III;
(o) drawing a desired volume of medicant from the vial into the medical syringe;
(p) displacing the switching component of said second fluid switching apparatus such to permit communication between fittings IV and VI;
(q) dispensing fluid from the medical syringe into the residue container to thereby prime the medical syringe prior to dispensing at a patient site;
(r) displacing the switching component of said first fluid switching apparatus to permit fluid communication between fittings II and III;
(s) dispensing fluid from the pre-filled flush syringe into the residue container to detachable connection between the needleless connector and fitting III and thereby also prime the pre-filled flush syringe prior to dispensing at a patient site; and
(t) detaching fitting III from the needleless connector only at a flushed site, wherefrom medication has been flushed, preparatory to delivery of the medical syringe and pre-filled flush syringe assembly to a site of use.

6. The method according to claim 5 whereby more than one dose of medicants is transferred from a single vial comprising the further steps of:
(u) affixing a fitting III of a new first fluid switching apparatus to the needleless connector to provide a new detachment site,
(v) repeating steps (e) and (f) for the new fluid switching apparatus;
(w) repeating steps (n) through (t).

7. A method for transferring medicants according to claim 1 comprising the further steps of:
(x) affixing a tubing set having a predetermined length to a body of said first fluid switching apparatus at an open site associated with said second pathway such that fitting II is distally displaced from the first fluid switching apparatus to a distance determined by the length of the tubing set.

8. A method for transferring medicants according to claim 1 comprising further assembly steps of:
(aa) providing an extension set having a male luer fitting (fitting VII) on one accessible site and an IV container on the other end and is otherwise closed;
(bb) affixing said needleless connector between fitting V and fitting VII, thereby providing a detachable connection between the needleless connector and fitting VII;
(cc) acquiring and affixing a medical syringe to fitting I;
(dd) acquiring and affixing a pre-filled flush syringe to fitting II; and
(ee) acquiring and securely affixing a vial containing a medicant to be transferred to fitting VI thereby providing a closed system comprising combined contents of said first and second kits.

9. The method for transferring medicants according to claim 8, if the medicant in the vial requires added liquid for reconstitution of lyophilized material, comprising further steps of:
(ff) displacing the switching component of said first fluid switching apparatus such to permit communication between fittings I and III;
(gg) displacing the switching component of said second fluid switching apparatus such to permit communication between fittings IV and VI;
(hh) drawing a predetermined volume of gas from the vial into the medication syringe to assure a negative transfer pressure;
(ii) displacing the switching component of said first fluid switching apparatus to permit fluid communication between fittings II and III;
(jj) dispensing a predetermined volume of liquid from the pre-filled flush syringe into the vial.

10. The method for transferring medicants according to claim 9 comprising further steps of:
(kk) displacing the switching component of said first fluid switching apparatus such to permit communication between fittings I and III;
(ll) drawing a desired volume of medicant from the vial into the medical syringe;
(mm) displacing the switching component of said second fluid switching apparatus to permit fluid communication between fittings IV and V;
(nn) dispensing fluid from the medical syringe into the extension set through fitting VII.
(oo) displacing the switching component of said first fluid switching apparatus such to permit communication between fittings I and III;
(pp) dispensing a predetermined volume of fluid from the pre-filled flush syringe into the extension set through fitting VII to flush medicant to the extension set and IV container through the detachable connection at fitting VII; and
(qq) detaching fitting VII from the needleless connector only at a flushed site, wherefrom medication has been displaced.

11. The method according to claim 10 whereby more than one dose of medicants is transferred from a single vial comprising the further steps of:
(u) affixing a fitting VII of a new extension set and associated IV container to the needleless connector to provide a new detachment site,
(v) repeating steps (e) and (f) for the new fluid switching apparatus;
(w) repeating steps (aa) through (qq).

12. The method according to claim 10 wherein both providing steps comprise the same steps.

13. A medical convenience kit for assembling an apparatus for measuring, filling and dispensing medication and flush solutions through connections to IV sets and patient lines and catheters while improving safety and efficacy of such procedures by requiring fewer post-sterilization makes and breaks than like procedures performed with conventional components, by facilitating dispensing of flush solutions, by providing for flushing of IV set and patient line and catheter connecting fittings before breaking such connections, by providing a two syringe assembly which provides for selectively dispensing from either of the two syringes while obstructing fluid displacement from the other syringe, said kit comprising:
a fluid switching apparatus comprising a first fitting for connecting to a medication delivery syringe, a second fitting for connecting to a pre-filled flush syringe, and a third fitting for accessing a medication container to thereby communicate a predetermined volume of a medication into the medication delivery syringe and, alternatively, for connecting to an external port for the purpose of dispensing fluid from the apparatus;
said fluid switching apparatus further comprising three intersecting pathways and an associated displace-able switching component disposed for selectively obstructing and permitting fluid flow through each of said intersecting pathways, a first pathway being disposed between said first fitting and said displace-able switching component, said second pathway being disposed between said second fitting and said displace-able switching component and said third pathway being disposed between said third fitting and said displaceable switching component;
said displaceable switching component comprising structure which, when displaced to a first state, selectively provides a fluid communication pathway only from said first pathway to said third pathway and when displaced to a second selected state provides a fluid communication pathway only between said second pathway and said third pathway, but under no conditions, in any state, provides a fluid communication pathway between said first and second pathways; and
said third fitting comprising an assembly which provides the pathway through which fluid is externally communicated and further comprising an associated connecting geometry having needleless connector compatible construction.

14. The medical convenience kit according to claim 13 further comprising a connecting link affixed to said second fitting whereby an attached flush syringe is aligned with the medication delivery syringe such that both syringes may be grasped and fluid manually delivered from either syringe by a single hand.

15. The medical convenience kit according to claim 14 wherein said connecting link is a short extension set, connecting on one end to said second fitting and on the other end to a flush syringe.

16. The medical convenience kit according to claim 13 wherein said fluid switching apparatus is a two-way stopcock having a core which is displace-able to two fluid conducting states, a first state only interconnecting said first pathway with said third pathway and said second state only interconnecting said second pathway with said third pathway.

17. The medical convenience kit according to claim 13 wherein said fluid switching apparatus is a pressure actuated switch comprising an obstructing plug which is displaced to a first state to open a pathway via a pressure generated within a medical delivery syringe, affixed to the first fitting, and communicated to the first pathway to provide a communicating intermediate pathway from the first pathway to the third pathway thereby and to a second state to open a pathway via a pressure generated within a flush syringe, affixed to the second fitting, and communicated to the second pathway to provide a communicating intermediate pathway from the second pathway to the third pathway thereby.

18. The medical convenience kit according to claim 17 wherein said fluid switching a further comprises a memory component associated with said obstructing plug whereby energy is stored in said component when pressure is applied to said plug to displace the plug to the second state and the stored energy is released to displace the plug to close communications between the second pathway and the third pathway to thereby block reflux contamination of fluid along the second pathway.

19. The medical convenience kit according to claim 15 further comprising a clip, which is releasibly affixed to a medication delivery syringe and a flush syringe to thereby stabilize and hold both syringes in alignment for facile management by a single hand

20. A method for measuring, filling and dispensing medication and flush solutions through connections to IV sets and catheters while improving safety and efficacy of such procedures by requiring fewer post-sterilization makes and breaks than like procedures performed with conventional components, by facilitating dispensing of flush solutions, by providing for flushing of IV set and catheter connecting fittings before breaking such connections, by providing a two syringe assembly for selectively dispensing from either of the two syringes, said method comprising the following steps:
(a) providing a packaged, sterilized kit comprising:
a fluid switching apparatus comprising a first fitting for connecting to a medication delivery syringe, a second fitting through which connection is made to a dedicated flush syringe, and a third fitting for accessing a medication container to thereby communicate a predetermined volume of a medication into the medication delivery syringe and, alternatively, for connecting to an external port for the purpose of dispensing fluid from the apparatus;
said fluid switching apparatus further comprising three intersecting pathways and an associated displaceable switching component disposed for selectively obstructing and permitting fluid flow through each of said intersecting pathways, a first pathway being disposed between said first fitting and said displace-able switching component, said second pathway being disposed between said second fitting and said displaceable switching component and said third pathway being disposed between said third fitting and said displaceable switching component;
said displaceable switching component comprising structure which, when displaced to a first state, selectively provides a fluid communication pathway only from said first pathway to said third pathway and when displaced to a second selected state closes external access to the first pathway and provides a fluid communication pathway only between said second pathway and said third pathway; and
said third fitting comprising an assembly which provides a pathway through which fluid is externally communicated and further comprising an associated connecting geometry having needleless connector compatible construction.
(b) disposing said kit in a work area where assembled parts are not contaminated and, then, opening said kit for access to provided parts;
(c) displacing said switching component to provide a communicating pathway between said first and third pathways and affixing a medical delivery syringe to said first fitting;
(d) affixing a pre-filled flush syringe to said second fitting to provide a complete assembly;
(e) affixing a vial access device having a needleless connector to said third fitting;
(f) accessing a medication disposed in a predetermined vial through the vial access device;
(g) drawing a predetermined volume of medication into the medication syringe;
(h) displacing the switching component to provide a communicating pathway between said second and third pathways;
(i) dispensing a predetermined volume of flush solution through said third fitting and needleless connector affixed thereto; and
(j) disconnecting said third fitting from said needleless connector to provide a free assembly thereby.

21. A method according to claim 20 comprising the following additional steps of:
(k) providing a luer tip cap and releasibly affixing said tip cap to said third fitting following step (j) to provide a transportable assembly and
(l) transporting the transportable assembly to a site of use.

22. The method according to claim 21 comprising the next steps of:
(m) removing said tip cap from the transportable assembly and
(n) affixing said second fitting to a needleless IV port connector affixed to a patient line or catheter as a first make at the site of use.

23. The method according to claim 22 comprising the next steps of:
(o) displacing said switching component to the first state; and
(p) dispensing a predetermined volume of medication from the medication delivery syringe through the needleless connector.

24. The method according to claim 24 comprising the next steps of:
(q) displacing said switching component to the second state to close external access to contents of the medical syringe and dispensing a predetermined fluid volume from the flush syringe through the needleless connector.
(r) detaching the third fitting from the needleless connector as a single break at the site of use; and
(s) disposing of the residual transportable assembly according to institutional protocol.

25. A method according to claim 20 comprising the steps of:
(t) providing an IV solution container/IV set combination comprising a predetermined volume of IV solution in the bag and a needleless connector for fluid transfer;
(u) pre-priming the IV set prior to injection of medication; and
(v) affixing said third fitting to the needeless connector of the combination.

26. A method according to claim 25 comprising the steps of:
(w) displacing said switching component to the first state; and
(x) dispensing a predetermined volume of medication through the needleless connector of the combination.

27. A method according to claim 26 comprising the steps of:
(y) displacing said switching component to the second state; and
(z) dispensing a predetermined volume of flush solution through the needleless connector of the combination.

28. The method according to claim 27 comprising the steps of:
(aa) detaching the second fitting from the needleless connector, and
(bb) disposing of the residual complete assembly according to institutional protocol.

29. The method according to claim 28 comprising steps of:
(cc) further providing a connecting link affixed to said second fitting whereby an attached flush syringe is aligned with the medication delivery syringe whereby both syringes may be grasped and operated by a single hand to manually deliver fluid from either syringe thereby.

30. The method according to claim 29 comprising the steps of:
(dd) providing a clip; and
(ee) affixing the medical syringe and flush syringe to the clip following completion of steps (c) and (d), thereby facilitating holding both syringes and actuating either syringe with a single hand.
